(19) United States
(12) Patent Application Publication
(10) Pub. No.: US 2007/0197446 A1
(43) Pub. Date: Aug. 23, 2007

(54) MHC binding peptides and their uses

(75) Inventors: Nikolai Franz Gregor Schwabe, Oxford (GB); Catherine Elizabeth Napper, Witney (GB)

Correspondence Address: KED & ASSOCIATES, LLP, P.O. Box 221200, Chantilly, VA, 20153-1200, US

(73) Assignee: ProImmune Limited

(21) Appl. No.: 11/702,570

(22) Filed: Feb. 6, 2007

(30) Foreign Application Priority Data
Feb. 20, 2006 (GB) GB0603336.9
Apr. 24, 2006 (GB) GB0608138.4
May 19, 2006 (GB) GB0609896.6

(51) Int. Cl. A61K 38/10; C07K 7/08

(52) U.S. Cl. 514/15; 530/328

(57) ABSTRACT

The present invention is concerned with MHC binding peptides derived from the avian influenza virus H5N1 and in particular MHC class I restricted binding peptides from the Haemagglutinin 5 (H5) protein from this virus and their uses.

The present invention is concerned with MHC binding peptides derived from the avian influenza virus H5N1 and in particular MHC class I restricted binding peptides from the Haemagglutinin 5 (H5) protein from this virus and their uses.

Major Histocompatibility Complex (MHC) molecules, which are found on the cell surface in tissues, play an important role in presenting cellular antigens in the form of short linear peptides to T cells by interacting with T cell receptors (TCRs) present on the surface of T cells.

In the above interaction of the MHC molecule and the T cell, the T cell recognizes and binds to the peptide bound in the MHC binding groove and the surrounding area of the MHC binding groove itself. In order for a peptide to bind to an MHC molecule and therefore be able to cause an immune response in vivo it needs to fit the properties of the binding groove, which are also referred to as the “binding motif”, of the relevant MHC allele in question. This binding motif restricts the type of peptides that can bind to a given MHC allele. Binding peptides matching the binding motif of a given MHC allele are also referred to as “restricted” to that allele.

It is known that class I MHC molecules typically bind peptide fragments of 8 to 11 amino acid length in their binding groove formed between the MHC class I alpha1 and alpha2 domains. For Class II MHC molecules binding of the peptides occurs in the groove formed between the alpha1 and beta1 domains of the molecule, the length of binding peptide varies more widely, and these peptides are typically 15 to 25 amino acids long or even longer, e.g. up to 30 amino acids length.

Understanding which peptides in a protein sequence can bind to a given MHC molecule and cause a T cell immune response is of considerable interest for understanding cellular immunity and in order to design and monitor the effectiveness of immunotherapeutic products, such as vaccines. MHC binding peptides that cause T cell immune responses are also referred to as “T cell epitopes”.

The relevance of identifying MHC-binding peptides has also been described extensively in the literature, such as in U.S. Pat. No. 5,585,461 or in U.S. Pat. No. 6,733,973, which describe the discovery and uses of MHC class I restricted T cell epitopes for the tumour antigen MAGE-3 and cytomegalovirus (CMV), respectively. U.S. Pat. No. 5,585,461 and U.S. Pat. No. 6,733,973 are incorporated herein by reference.

The H5N1 strain of avian influenza has been recognized as a serious threat to human health by the World Health Organisation. H5N1 avian influenza usually only affects birds, but it is thought to have the ability to mutate and acquire genes from viruses infecting other mammalian species. It can infect humans who have almost no natural immunity to the virus with high mortality. If the H5N1 virus mutates to increase its infectiousness to humans, a severe global flu pandemic could ensue. Presently no vaccine is available to treat H5N1 influenza in humans. Understanding immune responses to avian influenza is therefore critical in order to develop effective human therapies in the future.

### THE INVENTION

We have used an MHC-peptide binding assay as described in our co-pending patent application GB0519029.3, which is incorporated herein by reference, to identify MHC-binding peptides from the avian influenza virus H5N1. To perform our binding assay we synthesized a library of overlapping 9-mer peptides, offset from one another by one amino acid for the first 102 amino acids of the H5 protein from H5N1 Genbank/Swissprot ID: A/Vietnam/1203/04, Accession No.: AAW80717:

| SEQ ID NO: 1: | |
|---|---|
| MEKIVLLFAI VSLVKSDQIC IGYHANNSTE QVDTIMEKNV | 60 |
| TVTHAQDILE KKHNGKLCDL | |
| DGVKPLILRD CSVAGWLLGN PMCDEFINVP EWSYIVEKAN | 120 |
| PVNDLCYPGD FNDYEELKHL | |
| LSRINHFEKI QIIPKSSWSS HEASLGVSSA CPYQGKSSFF | 180 |
| RNVVWLIKKN STYPTIKRSY | |
| NNTNQEDLLV LWGIHHPNDA AEQTKLYQNP TTYISVGTST | 240 |
| LNQRLVPRIA TRSKVNGQSG | |
| RMEFFWTILK PNDAINFESN GNFIAPEYAY KIVKKGDSTI | 300 |
| MKSELEYGNC NTKCQTPMGA | |
| INSSMPFHNI HPLTIGECPK YVKSNRLVLA TGLRNSPQRE | 360 |
| RRRKKRGLFG AIAGFIEGGW | |
| QGMVDGWYGY HHSNEQGSGY AADKESTQKA IDGVTNKVNS | 420 |
| IIDKMNTQFE AVGREFNNLE | |
| RRIENLNKKM EDGFLDVWTY NAELLVLMEN ERTLDFHDSN | 480 |
| VKNLYDKVRL QLRDNAKELG | |
| NGCFEFYHKC DNECMESVRN GTYDYPQYSE EARLKREEIS | 540 |
| GVKLESIGIY QILSIYSTVA | |
| SSLALAIMVA GLSLWMCSNG SLQCRICI | 568 |

The following known restricted T cell epitopes were used as controls in our MHC peptide binding assay to assess the binding of peptides to HLA-A*0101:

TABLE 1

Sequences of control peptides used

| | | | |
|---|---|---|---|
| | | | ELISA (Absorp- |
| | Control | Peptide | tion-Pass/Fail |
| SEQ ID NO: | Type | Sequence | Control) |

| | | | |
|---|---|---|---|
| SEQ ID NO: 2 | Positive | YSEHPFTSQY | 0.58 |
| | Control | | |
| SEQ ID NO: 3 | Pass/Fail | AAGIGILTV | 0 |
| | Control | | |

The following known restricted T cell epitopes were used as controls in our MHC peptide binding assay to assess the binding of peptides to HLA-A*0201:

TABLE 2

Sequences of control peptides used

| | | | |
|---|---|---|---|
| | Control | Peptide | ELISA (Absorption- |
| SEQ ID NO: | Type | Sequence | Pass/Fail Control) |

| | | | |
|---|---|---|---|
| SEQ ID NO: 4 | Positive | GILGFVFTL | 1.20 |
| | Control | | |
| SEQ ID NO: 3 | Pass/Fail | AAGIGILTV | 0 |
| | Control | | |

The following known restricted T cell epitopes were used as controls in our MHC peptide binding assay to assess the binding of peptides to HLA-A*0301:

TABLE 3

Sequences of control peptides used

| | | | |
|---|---|---|---|
| | Control | Peptide | ELISA (Absorption- |
| SEQ ID NO: | Type | Sequence | Pass/Fail Control) |

| | | | |
|---|---|---|---|
| SEQ ID NO: 5 | Positive | ILRGSVAHK | 1.19 |
| | Control | | |
| SEQ ID NO: 3 | Pass/Fail | AAGIGILTV | 0 |
| | Control | | |

The following known restricted T cell epitopes were used as controls in our MHC peptide binding assay to assess the binding of peptides to HLA-B*0702:

TABLE 4

Sequences of control peptides used

| | | | |
|---|---|---|---|
| | | | ELISA (Absorp- |
| | Control | Peptide | tion-Pass/Fail |
| SEQ ID NO: | Type | Sequence | Control) |

| | | | |
|---|---|---|---|
| SEQ ID NO: 6 | Positive | TPRVTGGGAM | 0.88 |
| | Control | | |
| SEQ ID NO: 3 | Pass/Fail | AAGIGILTV | 0 |
| | Control | | |

The following known T cell epitopes were used as controls in our MHC peptide binding assay to assess the binding of peptides to HLA-A*0801:

TABLE 5

Sequences of control peptides used

| | | | |
|---|---|---|---|
| | Control | Peptide | ELISA (Absorption- |
| SEQ ID NO: | Type | Sequence | Pass/Fail Control) |

| | | | |
|---|---|---|---|
| SEQ ID NO: 7 | Positive | RAKFKQLL | 0.73 |
| | Control | | |
| SEQ ID NO: 3 | Pass/Fail | AAGIGILTV | 0 |
| | Control | | |

The following known restricted T cell epitopes were used as controls in our MHC peptide binding assay to assess the binding of peptides to HLA-B*3501:

TABLE 6

Sequences of control peptides used

| | | |
|---|---|---|
| | | ELISA (Absorp- |
| Control | Peptide | tion-Pass/Fail |

| SEQ ID NO: | Type | Sequence | Control) |
|---|---|---|---|
| SEQ ID NO: 8 | Positive | HPVGEADYFEY | 0.55 |
| | Control | | |
| SEQ ID NO: 3 | Pass/Fail | AAGIGILTV | 0 |
| | Control | | |

We have found that 2 9-mer peptides from the H5 protein in H5N1 (SEQ ID NO: 1) bind with an efficiency to HLA-A*0101 that is similar as or better than the known T cell epitope that has been used as a pass/fail control SEQ ID NO: 3.

These peptides of the invention are listed in Table 7 below and are listed in single letter amino acid nomenclature:

TABLE 7

Sequences of peptides of the invention binding
to HLA-A*0101

| | Position of | | ELISA (Ab- |
|---|---|---|---|
| | first amino acid | | sorption- |
| | in H5 protein | Peptide | Pass/Fail |
| SEQ ID NO: | (SEQ ID NO: 1) | Sequence | Control) |
| SEQ ID NO: 9 | 15 | KSDQICIGY | 0.47 |
| SEQ ID NO: 10 | 86 | FINVPEWSY | 0.16 |

Further we have found that 16 9-mer peptides from the H5 protein in H5N1 (SEQ ID NO: 1) bind with an efficiency to HLA-A*0201 that is similar as or better than a known T cell epitope that has been used as a pass/fail control.

These peptides of the invention are listed in Table 8 below and are listed in single letter amino acid nomenclature:

TABLE 8

Sequences of peptides of the invention binding
to HLA-A*0201

| | | | |
|---|---|---|---|
| | Position of | | |
| | first amino | | ELISA |
| | acid in H5 | | (Absorption- |
| | protein | Peptide | Pass/Fail |
| SEQ ID NO: | (SEQ ID NO: 1) | Sequence | Control) |

| | | | |
|---|---|---|---|
| SEQ ID NO: 11 | 5 | VLLFAIVSL | 0.47 |
| SEQ ID NO: 12 | 6 | LLFAIVSLV | 1.06 |
| SEQ ID NO: 13 | 8 | FAIVSLVKS | 0.07 |
| SEQ ID NO: 14 | 9 | AIVSLVKSD | 0.43 |
| SEQ ID NO: 15 | 10 | IVSLVKSDQ | 0.04 |
| SEQ ID NO: 16 | 12 | SLVKSDQIC | 0.09 |
| SEQ ID NO: 17 | 24 | HANNSTEQV | 0.43 |
| SEQ ID NO: 18 | 34 | TIMEKNVTV | 0.99 |
| SEQ ID NO: 19 | 35 | IMEKNVTVT | 0.02 |
| SEQ ID NO: 20 | 36 | MEKNVTVTH | −0.02 |
| SEQ ID NO: 21 | 45 | AQDILEKKH | 0.15 |
| SEQ ID NO: 22 | 56 | KLCDLDGVK | 0.20 |
| SEQ ID NO: 23 | 66 | LILRDCSVA | 0.57 |
| SEQ ID NO: 24 | 81 | PMCDEFINV | 0.98 |
| SEQ ID NO: 25 | 88 | NVPEWSYIV | 1.03 |
| SEQ ID NO: 26 | 94 | YIVEKANPV | 1.02 |

Further we have found 4 9-mer peptides from the H5 protein, SEQ ID NO: 1, which bind with an efficiency to HLA-A*0301 that is similar as or better than a known T cell epitope that has been used as a pass/fail control. These peptides of the invention are listed in Table 9 below and are listed in single letter amino acid nomenclature:

TABLE 9

Sequences of peptides of the invention binding
to HLA-A*0301

| | | | |
|---|---|---|---|
| | Position of | | |
| | first amino | | ELISA |
| | acid in H5 | | (Absorption- |
| | protein | Peptide | Pass/Fail |
| SEQ ID NO: | (SEQ ID NO: 1) | Sequence | Control) |

| | | | |
|---|---|---|---|
| SEQ ID NO: 9 | 15 | KSDQICIGY | 0.05 |
| SEQ ID NO: 27 | 39 | NVTVTHAQD | −0.04 |
| SEQ ID NO: 28 | 48 | ILEKKHNGK | −0.01 |
| SEQ ID NO: 22 | 56 | KLCDLDGVK | 1.16 |

Further we have found 4 9-mer peptides from the H5 protein, SEQ ID NO: 1, which bind with an efficiency to HLA-B*0702 that is similar as or better than that of a known T cell epitope that has been used as a pass/fail control. These peptides of the invention are listed in Table 10 below and are listed in single letter amino acid nomenclature:

TABLE 10

Sequences of peptides binding to HLA-B*0702

| | | | |
|---|---|---|---|
| | Position of | | |
| | first amino | | ELISA |
| | acid in H5 | | (Absorption- |
| | protein | Peptide | Pass/Fail |
| SEQ ID NO: | (SEQ ID NO: 1) | Sequence | Control) |

| | | | |
|---|---|---|---|
| SEQ ID NO: 29 | 13 | LVKSDQICI | −0.06 |
| SEQ ID NO: 18 | 34 | TIMEKNVTV | 0.00 |
| SEQ ID NO: 30 | 67 | ILRDCSVAG | 0.03 |
| SEQ ID NO: 26 | 94 | YIVEKANPV | 0.11 |

Further we have found 14 9-mer peptides from the H5 protein, SEQ ID NO: 1, which bind with an efficiency to HLA-B*0801 that is similar as or better than the known T cell epitope that has been used as a pass/fail control SEQ ID NO: 3. These peptides of the invention are listed in Table 11 below and are listed in single letter amino acid nomenclature:

TABLE 11

Sequences of peptides of the invention binding
to HLA-A*0801

| | Position of | | |
|---|---|---|---|
| | first amino | | ELISA |
| | acid in H5 | | (Absorption- |
| | protein | Peptide | Pass/Fail |
| SEQ ID NO: | (SEQ ID NO: 1) | Sequence | Control) |
| SEQ ID NO: 31 | 11 | VSLVKSDQI | 0.36 |
| SEQ ID NO: 32 | 33 | DTIMEKNVT | 0.08 |
| SEQ ID NO: 18 | 34 | TIMEKNVTV | 0.80 |
| SEQ ID NO: 27 | 39 | NVTVTHAQD | 0.08 |
| SEQ ID NO: 33 | 42 | VTHAQDILE | 0.31 |
| SEQ ID NO: 34 | 47 | DILEKKHNG | 0.53 |
| SEQ ID NO: 28 | 48 | ILEKKHNGK | 0.95 |
| SEQ ID NO: 35 | 64 | KPLILRDCS | 0.10 |
| SEQ ID NO: 36 | 65 | PLILRDCSV | 0.65 |
| SEQ ID NO: 23 | 66 | LILRDCSVA | 0.07 |
| SEQ ID NO: 37 | 73 | VAGWLLGNP | 0.04 |
| SEQ ID NO: 38 | 76 | WLLGNPMCD | 0.03 |
| SEQ ID NO: 39 | 79 | GNPMCDEFI | 0.04 |
| SEQ ID NO: 26 | 94 | YIVEKANPV | 0.32 |

Further we have found that 8 9-mer peptides from the H5 protein in H5N1 (SEQ ID NO: 1) bind with an efficiency to HLA-B*3501 that is similar as or better than the known T cell epitope that has been used as a pass/fail control SEQ ID NO: 3. These peptides of the invention are listed in Table 12 below and are listed in single letter amino acid nomenclature:

TABLE 12

Sequences of peptides of the invention binding
to HLA-B*3501

| | | | |
|---|---|---|---|
| | Position of | | |
| | first amino | | ELISA |
| | acid in H5 | | (Absorption- |
| | protein | Peptide | Pass/Fail |
| SEQ ID NO: | (SEQ ID NO: 1) | Sequence | Control) |

| | | | |
|---|---|---|---|
| SEQ ID NO: 40 | 7 | LFAIVSLVK | 0.10 |
| SEQ ID NO: 16 | 12 | SLVKSDQIC | −0.03 |
| SEQ ID NO: 29 | 13 | LVKSDQICI | 0.04 |
| SEQ ID NO: 17 | 24 | HANNSTEQV | 0.43 |
| SEQ ID NO: 41 | 51 | KKHNGKLCD | −0.05 |
| SEQ ID NO: 42 | 74 | AGWLLGNPM | −0.06 |
| SEQ ID NO: 43 | 78 | LGNPMCDEF | 0.35 |
| SEQ ID NO: 10 | 86 | FINVPEWSY | 0.57 |

In its first aspect the invention therefore concerns one or more of the isolated peptides selected from the group consisting of:

| | |
|---|---|
| KSDQICIGY, | (SEQ ID NO: 9) |
| FINVPEWSY, | (SEQ ID NO: 10) |
| VLLFAIVSL, | (SEQ ID NO: 11) |
| LLFAIVSLV, | (SEQ ID NO: 12) |
| FAIVSLVKS, | (SEQ ID NO: 13) |
| AIVSLVKSD, | (SEQ ID NO: 14) |
| IVSLVKSDQ, | (SEQ ID NO: 15) |
| SLVKSDQIC, | (SEQ ID NO: 16) |
| HANNSTEQV, | (SEQ ID NO: 17) |
| TIMEKNVTV, | (SEQ ID NO: 18) |
| IMEKNVTVT, | (SEQ ID NO: 19) |
| MEKNVTVTH, | (SEQ ID NO: 20) |
| AQDILEKKH, | (SEQ ID NO: 21) |
| KLCDLDGVK, | (SEQ ID NO: 22) |
| LILRDCSVA, | (SEQ ID NO: 23) |
| PMCDEFINV, | (SEQ ID NO: 24) |
| NVPEWSYIV, | (SEQ ID NO: 25) |
| YIVEKANPV, | (SEQ ID NO: 26) |
| NVTVTHAQD, | (SEQ ID NO: 27) |
| ILEKKHNGK, | (SEQ ID NO: 28) |
| LVKSDQICI, | (SEQ ID NO: 29) |
| ILRDCSVAG, | (SEQ ID NO: 30) |
| VSLVKSDQI, | (SEQ ID NO: 31) |
| DTIMEKNVT, | (SEQ ID NO: 32) |
| VTHAQDILE, | (SEQ ID NO: 33) |
| DILEKKHNG, | (SEQ ID NO: 34) |
| KPLILRDCS, | (SEQ ID NO: 35) |
| PLILRDCSV, | (SEQ ID NO: 36) |
| VAGWLLGNP, | (SEQ ID NO: 37) |
| WLLGNPMCD, | (SEQ ID NO: 38) |
| GNPMCDEFI, | (SEQ ID NO: 39) |
| LFAIVSLVK, | (SEQ ID NO: 40) |
| KKHNGKLCD, | (SEQ ID NO: 41) |
| AGWLLGNPM, | (SEQ ID NO: 42) |
| and | |
| LGNPMCDEF. | (SEQ ID NO: 43) |

The binding assay was carried out according the methods described in the applicant's co-pending British patent application GB0519029.3, measuring the extent of assembly of the peptides at a known concentration with the HLA alpha and beta chains over 24 hours. The extent of assembly was measured quantitatively for each peptide using a solid phase binding assay (ELISA) with a conformational antibody that only detects fully assembled MHC complexes. The ELISA absorption readings were taken for all peptides to be tested and the respective control peptides, which are known T cell epitopes, i.e. they are known to elicit T cell immune responses in humans. SEQ ID NO: 2 is a peptide binding with high efficiency to HLA-A*0101, SEQ ID NO: 4 is a peptide binding with high efficiency to HLA-A*0201, SEQ ID NO: 5 is a peptide binding with high efficiency to HLA-A*0301, SEQ ID NO: 6 is a peptide binding with high efficiency to HLA-B*0702, SEQ ID NO: 7 is a peptide binding with high efficiency to HLA-B*0801, SEQ ID NO: 8 is a peptide binding with high efficiency to HLA-B*3501, and SEQ ID NO: 3 is a peptide binding with intermediate efficiency to HLA-A*0201 which is used as a pass/fail control in all assays. SEQ ID NO: 3 was chosen as a pass/fail control since it is a relatively weak binding peptide on the one hand, but it has still been confirmed as a T cell epitope on the other. The extent of the binding of SEQ ID NO: 3 was always assessed for its binding to HLA-A*0201. The peptides of the invention were selected based on their ability to bind with similar or better efficiency to the MHC alleles as stated in the tables above than the control peptide is able to bind to HLA-A*0201. Although binding of the pass/fail control peptide was therefore measured with a different MHC allele than for the peptides to be tested in the assay, the use of the pass/fail control peptide for this purpose was validated: the same molar amounts were used for each component in the assay irrespective of the MHC allele studied and it was ascertained that the detection system renders substantially identical conformational signals when identical molar amounts of purified conformational MHC complexes are used for each of HLA-A*0101, HLA-A*0201, HLA-A*0301, HLA-B*0702, HLA-B*0801, and HLA-B*3501. The tables above show the ELISA absorption readings taken for each peptide with the absorption reading for the control peptide subtracted.

The peptides of the invention are useful in a number of ways:

They may be used as isolated peptides, e.g. in the form of synthetic peptides in prophylactic or therapeutic vaccines to treat or vaccinate against influenza infection. They are useful in this way when bound to HLA-A1, A2, A3, B7, B8, and HLA-B35 complexes, as applicable, and on their own. Alternatively they may be segments in larger protein based vaccine constructs. They may be administered in the form of one or more of the isolated peptides of the invention or lipopeptide vaccine, optionally with an adjuvant such as described in U.S. Pat. No. 6,733,973.

Obtaining the isolated peptides of the invention is well known in the art, e.g. by solid phase peptide synthesis, which is e.g. offered as a routine service to researchers by many companies today, such as Sigma-Genosys, Woodlands, Tex., USA.

The invention further concerns DNA or RNA sequences encoding one or more of the peptides of the invention. These DNA or RNA sequences may be used in DNA or RNA constructs for heterologous expression in a prokaryotic host cell, such as E. coli., or a eukaryotic host cell, such as an insect cell or mammalian cell. The constructs may be viral vector based constructs such as vaccinia or pox vector constructs, which may be used as vaccines or other immunotherapies to treat or vaccinate against influenza infection. The actual sequences of such coding DNA or RNA sequences may be based on the original coding sequences of the H5N1 virus studied here and may be adapted based on the host cell in which these sequences are to be expressed. Back-translation from protein to DNA and RNA coding sequences is documented extensively in the literature. Such coding DNA or RNA sequences may offer additional design options, such as selecting codon-optimised sequences for the expression in the host of interest or other codon choices, e.g. suited to the restriction sites in the expression system used.

In a third aspect thereof the invention therefore concerns a DNA or RNA sequence encoding one or more of the peptides of the invention and a vector comprising such a DNA or RNA sequence.

The construction of vaccines based on the peptides of the invention or RNA, DNA coding therefore is described in some detail in U.S. Pat. No. 6,733,973.

In another alternative embodiment the peptides may be incorporated in an oligomeric MHC-peptide complex, such as in fluorescent labelled MHC pentamers as described in WO04018520, which is incorporated herein by reference, or MHC tetramers which may be formed by coupling monomeric MHC-peptide complexes incorporating the peptides of the invention to a multivalent entity at a specific attachment site, as described in U.S. Pat. No. 5,635,363, which are useful for labelling, detecting and isolating antigen-specific T cells. Such MHC multimers are consequently useful for immune monitoring of single-antigen specific T cells that are reactive to the peptides of the invention in patients.

In a fourth aspect therefore the invention concerns an MHC-peptide complex comprising one or more of the peptides of the invention and in a fifth aspect the invention concerns an oligomeric MHC-peptide complex comprising one or more of the peptides of the invention.

In yet another embodiment the binding peptides of the invention can be used to raise polyclonal or monoclonal antibodies against the peptides of the invention, which may be useful for diagnosing influenza infection or presentation of the specific peptide on a cell. The detection of such specific peptide presentation can be very important when tracking the distribution of MHC complexes presenting a particular peptide in vivo, e.g. in the context of a vaccination protocol where the vaccination occurs through a particular administration route and/or location and is designed to effect presentation of a specific peptide. In this case it is often required that the MHC-peptide presentation is then translated to a specific site of action in the vaccinated individual's tissues. Monitoring the effectiveness of such translocation of antigen presentation is possible by using binding of antibodies that are specific to the MHC-peptide combination of interest, whereby the antibodies themselves are labelled to enable visualisation, e.g. through a fluorescent or radioactive label, which may be traceable in vivo. Further such antibodies may also be used in antibody-based therapy to fight influenza infection, e.g. as normal complement-fixing antibodies or radiolabelled antibodies that are used to kill infected cell with an effective dose of radiation. The production of radiolabelled monoclonal antibodies for treating cancer in a similar way is described in detail in WO9300927, which is incorporated herein by reference. Although WO9300927 describes the use of radiolabelled monoclonal antibodies for treating cancer the methodology is readily translated to the treatment of infectious diseases.

In a sixth aspect the invention therefore comprises an antibody specific to one or more of the peptides of the invention or specific to an MHC-peptide complex comprising one or more of the peptides of the invention.

In a further embodiment of the invention cytolytic T cell lines or clones that are reactive to the peptides of the invention may be raised. Raising such T cell lines may be effected as described in U.S. Pat. No. 5,585,461 or U.S. Pat. No. 6,733,973. Such antigen-specific T cell lines can also be generated by incorporating the peptides of the invention in MHC pentamers, such as described in the applicant's WO04018520. Such MHC pentamers can be coupled to paramagnetic beads, such as Dynabeads®, which are useful for isolating peptide-specific T cells. Cells isolated in this manner can be expanded in culture and are suitable for the therapeutic protocols described in. Such cell lines are further useful as positive controls for monitoring immune responses against the peptides of interest in patients. Alternatively they may be useful in a therapeutic application where it is desirable to administer T cells specific to one or more peptides of the invention to a patient either autologously or allogeneically, e.g. in order to increase their immune response to the antigen in question, such as described in U.S. Pat. No. 6,733,973.

In a seventh aspect the invention therefore concerns a cytolytic T cell line or clone specific to one or more of the peptides of the invention.

Alternatively the peptides of the invention may be administered in the form of a cellular vaccine through the administration of autologous or allogeneic antigen presenting cells or dendritic cells that have been treated in vitro so as to present one or more of the peptides of the invention on their surface. The preparation of such antigen presenting cells is described in U.S. Pat. No. 6,733,973.

In an eighth aspect the invention therefore concerns an isolated dendritic cell presenting one or more of the peptides of the invention.

In a ninth aspect the invention concerns pharmaceutical composition comprising one or more selected from the group consisting of one or more of the peptides of the invention, a DNA or RNA sequence of the invention, a vector of the invention, an MHC-peptide complex of the invention, an oligomeric MHC-peptide complex the invention, an antibody of the invention, a cytolytic T cell line of the invention, and a dendritic cell of the invention.

The pharmaceutical composition of the invention can be used for treating influenza infection or be administered for prophylactic vaccination.

Pharmaceutical compositions comprising the peptides are useful for, e.g. parenteral administration, i.e. subcutaneously, intramuscularly or intravenously, but not limited thereto.

The compositions for parenteral administration may for example comprise the active ingredients dissolved or suspended in an acceptable carrier, preferably an aqueous carrier. A variety of aqueous carriers can be used, e.g. buffered water, 0.4% saline, 0.3% glycine and the like. These solutions are sterile and generally free of particulate matter and may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride, sodium lactate, etc. The concentration of the active ingredients in these formulations can vary widely, i.e. in the case of soluble matter from less than about 1 pg/ml, usually at least about 0.1 mg/ml to as much as 10-100 mg/ml and will be selected primarily based on fluid volumes, viscosities, etc. in accordance with the particular mode of administration selected.

In a tenth aspect the invention concerns a diagnostic composition comprising a label and one or more selected from the group consisting of one or more of the peptides of the invention, a DNA or RNA sequence of the invention, a vector of the invention, an MHC-peptide complex of the invention, an oligomeric MHC-peptide complex the invention, an antibody of the invention, a cytolytic T cell line of the invention, and a dendritic cell of the invention.

The diagnostic compositions of the invention can be used to detect, characterise and monitor influenza infections in individuals, detect cells that are presenting or are reactive to the peptides of the invention in a cell population and generally assess the immune competence of individuals relative to influenza infection. For this purpose such reagents may be provided with a label, such as a radioactive or fluorescent label as described in U.S. Pat. No. 6,733,973 and WO04018520.

It will be understood that the foregoing examples are merely given to illustrate the invention and the invention is not limited thereto. Other uses of the peptide sequences described herein will be evident to the skilled practitioner.

1. An isolated peptide selected from the group consisting of:

| | |
|---|---|
| KSDQICIGY, | (SEQ ID NO: 9) |
| FINVPEWSY, | (SEQ ID NO: 10) |
| VLLFAIVSL, | (SEQ ID NO: 11) |
| LLFAIVSLV, | (SEQ ID NO: 12) |
| FAIVSLVKS, | (SEQ ID NO: 13) |
| AIVSLVKSD, | (SEQ ID NO: 14) |
| IVSLVKSDQ, | (SEQ ID NO: 15) |
| SLVKSDQIC, | (SEQ ID NO: 16) |
| HANNSTEQV, | (SEQ ID NO: 17) |
| TIMEKNVTV, | (SEQ ID NO: 18) |
| IMEKNVTVT, | (SEQ ID NO: 19) |
| MEKNVTVTH, | (SEQ ID NO: 20) |
| AQDILEKKH, | (SEQ ID NO: 21) |
| KLCDLDGVK, | (SEQ ID NO: 22) |
| LILRDCSVA, | (SEQ ID NO: 23) |
| PMCDEFINV, | (SEQ ID NO: 24) |
| NVPEWSYIV, | (SEQ ID NO: 25) |
| YIVEKANPV, | (SEQ ID NO: 26) |
| NVTVTHAQD, | (SEQ ID NO: 27) |
| ILEKKHNGK, | (SEQ ID NO: 28) |
| LVKSDQICI, | (SEQ ID NO: 29) |
| ILRDCSVAG, | (SEQ ID NO: 30) |
| VSLVKSDQI, | (SEQ ID NO: 31) |
| DTIMEKNVT, | (SEQ ID NO: 32) |
| VTHAQDILE, | (SEQ ID NO: 33) |
| DILEKKHNG, | (SEQ ID NO: 34) |
| KPLILRDCS, | (SEQ ID NO: 35) |
| PLILRDCSV, | (SEQ ID NO: 36) |
| VAGWLLGNP, | (SEQ ID NO: 37) |
| WLLGNPMCD, | (SEQ ID NO: 38) |
| GNPMCDEFI, | (SEQ ID NO: 39) |
| LFAIVSLVK, | (SEQ ID NO: 40) |
| KKHNGKLCD, | (SEQ 1D NO: 41) |
| AGWLLGNPM, | (SEQ ID NO: 42) |
| and | |
| LGNPMCDEF. | (SEQ ID NO: 43) |

2. A DNA or RNA sequence encoding one or more of the peptides of claim 1.

3. A vector comprising a DNA or RNA sequence of claim 2.

4. An MHC-peptide complex comprising one or more of the peptides of claim 1.

5. An oligomeric MHC-peptide complex comprising one or more of the peptides of claim 1.

6. An antibody specific to one or more of the peptides of claim 1 or an MHC-peptide complex comprising one or more of the peptides of claim 1.

7. A cytolytic T cell line or clone specific to one or more of the peptides of claim 1.

8. A dendritic cell primed with one or more of the peptide of claim 1.

9. A pharmaceutical composition comprising one or more selected from the group consisting of one or more of the peptides of claim 1, a DNA or RNA sequence of claim 2, a vector of claim 3, an MHC-peptide complex of claim 4, an oligomeric MHC-peptide complex of claim 5, an antibody of claim 6, a cytolytic T cell line or clone of claim 7, and a dendritic cell of claim 8.

10. A diagnostic composition comprising a label and one or more selected from the group consisting of one or more of the peptides of claim 1, a DNA or RNA sequence of claim 2, a vector of claim 3, an MHC-peptide complex of claim 4, an oligomeric MHC-peptide complex of claim 5, an antibody of claim 6, a cytolytic T cell line or clone of claim 7, and a dendritic cell of claim 8.


SEQUENCE LISTING (43 sequences)

SEQ ID NO: 1 - protein, 568 aa, H5N1
MEKIVLLFAIVSLVKSDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 2 - protein, 10 aa, Artificial Sequence
  T cell epitope
YSEHPFTSQY

SEQ ID NO: 3 - protein, 9 aa, Artificial Sequence
  T cell epitope
AAGIGILTV

SEQ ID NO: 4 - protein, 9 aa, Artificial Sequence
  T cell epitope
GILGFVFTL

SEQ ID NO: 5 - protein, 9 aa, Artificial Sequence
  T cell epitope
ILRGSVAHK

SEQ ID NO: 6 - protein, 10 aa, Artificial Sequence
  T cell epitope
TPRVTGGGAM

SEQ ID NO: 7 - protein, 8 aa, Artificial Sequence
  T cell epitope
RAKFKQLL

SEQ ID NO: 8 - protein, 11 aa, Artificial Sequence
  T cell epitope
HPVGEADYFEY

SEQ ID NO: 9 - protein, 9 aa, Artificial Sequence
  9-mer peptide from the H5 protein in H5N1
KSDQICIGY

SEQ ID NO: 10 - protein, 9 aa, Artificial Sequence
  9-mer peptide from the H5 protein in H5N1
FINVPEWSY

SEQ ID NO: 11 - protein, 9 aa, Artificial Sequence
  9-mer peptide from the H5 protein in H5N1
VLLFAIVSL

SEQ ID NO: 12 - protein, 9 aa, Artificial Sequence
  9-mer peptide from the H5 protein in H5N1
LLFAIVSLV

SEQ ID NO: 13 - protein, 9 aa, Artificial Sequence
  9-mer peptide from the H5 protein in H5N1
FAIVSLVKS

SEQ ID NO: 14 - protein, 9 aa, Artificial Sequence
  9-mer peptide from the H5 protein in H5N1
AIVSLVKSD

SEQ ID NO: 15 - protein, 9 aa, Artificial Sequence
  9-mer peptide from the H5 protein in H5N1
IVSLVKSDQ

SEQ ID NO: 16 - protein, 9 aa, Artificial Sequence
  9-mer peptide from the H5 protein in H5N1
SLVKSDQIC

SEQ ID NO: 17 - protein, 9 aa, Artificial Sequence
  9-mer peptide from the H5 protein in H5N1
HANNSTEQV

SEQ ID NO: 18 - protein, 9 aa, Artificial Sequence
  9-mer peptide from the H5 protein in H5N1
TIMEKNVTV

SEQ ID NO: 19 - protein, 9 aa, Artificial Sequence
  9-mer peptide from the H5 protein in H5N1
IMEKNVTVT

SEQ ID NO: 20 - protein, 9 aa, Artificial Sequence
  9-mer peptide from the H5 protein in H5N1
MEKNVTVTH

SEQ ID NO: 21 - protein, 9 aa, Artificial Sequence
  9-mer peptide from the H5 protein in H5N1
AQDILEKKH

SEQ ID NO: 22 - protein, 9 aa, Artificial Sequence
  9-mer peptide from the H5 protein in H5N1
KLCDLDGVK

SEQ ID NO: 23 - protein, 9 aa, Artificial Sequence
  9-mer peptide from the H5 protein in H5N1
LILRDCSVA

SEQ ID NO: 24 - protein, 9 aa, Artificial Sequence
  9-mer peptide from the H5 protein in H5N1
PMCDEFINV

SEQ ID NO: 25 - protein, 9 aa, Artificial Sequence
  9-mer peptide from the H5 protein in H5N1
NVPEWSYIV

SEQ ID NO: 26 - protein, 9 aa, Artificial Sequence
  9-mer peptide from the H5 protein in H5N1
YIVEKANPV

SEQ ID NO: 27 - protein, 9 aa, Artificial Sequence
  9-mer peptide from the H5 protein in H5N1
NVTVTHAQD

SEQ ID NO: 28 - protein, 9 aa, Artificial Sequence
  9-mer peptide from the H5 protein in H5N1
ILEKKHNGK

SEQ ID NO: 29 - protein, 9 aa, Artificial Sequence
  9-mer peptide from the H5 protein in H5N1
LVKSDQICI

SEQ ID NO: 30 - protein, 9 aa, Artificial Sequence
  9-mer peptide from the H5 protein in H5N1
ILRDCSVAG

SEQ ID NO: 31 - protein, 9 aa, Artificial Sequence
  9-mer peptide from the H5 protein in H5N1
VSLVKSDQI

SEQ ID NO: 32 - protein, 9 aa, Artificial Sequence
  9-mer peptide from the H5 protein in H5N1
DTIMEKNVT

SEQ ID NO: 33 - protein, 9 aa, Artificial Sequence
  9-mer peptide from the H5 protein in H5N1
VTHAQDILE

SEQ ID NO: 34 - protein, 9 aa, Artificial Sequence
  9-mer peptide from the H5 protein in H5N1
DILEKKHNG

SEQ ID NO: 35 - protein, 9 aa, Artificial Sequence
  9-mer peptide from the H5 protein in H5N1
KPLILRDCS

SEQ ID NO: 36 - protein, 9 aa, Artificial Sequence
  9-mer peptide from the H5 protein in H5N1
PLILRDCSV

SEQ ID NO: 37 - protein, 9 aa, Artificial Sequence
  9-mer peptide from the H5 protein in H5N1
VAGWLLGNP

SEQ ID NO: 38 - protein, 9 aa, Artificial Sequence
  9-mer peptide from the H5 protein in H5N1
WLLGNPMCD

SEQ ID NO: 39 - protein, 9 aa, Artificial Sequence
  9-mer peptide from the H5 protein in H5N1
GNPMCDEFI

SEQ ID NO: 40 - protein, 9 aa, Artificial Sequence
  9-mer peptide from the H5 protein in H5N1
LFAIVSLVK

SEQ ID NO: 41 - protein, 9 aa, Artificial Sequence
  9-mer peptide from the H5 protein in H5N1
KKHNGKLCD

SEQ ID NO: 42 - protein, 9 aa, Artificial Sequence
  9-mer peptide from the H5 protein in H5N1
AGWLLGNPM

SEQ ID NO: 43 - protein, 9 aa, Artificial Sequence
  9-mer peptide from the H5 protein in H5N1
LGNPMCDEF